(19) United States
(12) Patent Application Publication
(10) Pub. No.: US 2017/0172431 A1
(43) Pub. Date: Jun. 22, 2017

(54) METHOD AND APPARATUS OF CALCULATING BLOOD PRESSURE

(71) Applicant: Samsung Electronics Co., Ltd., Suwon-si (KR)

(72) Inventors: Younho KIM, Hwaseong-si (KR); Yongjoo KWON, Yongin-si (KR); Ui Kun KWON, Hwaseong-si (KR); Seungwoo NOH, Seongnam-si (KR); Sangyun PARK, Hwaseong-si (KR)

(73) Assignee: Samsung Electronics Co., Ltd., Suwon-si (KR)

(21) Appl. No.: 15/250,996

(22) Filed: Aug. 30, 2016

(30) Foreign Application Priority Data
Dec. 18, 2015 (KR) 10-2015-0181443

(51) Int. Cl. A61B 5/021; A61B 5/11; A61B 5/00

(52) U.S. Cl. CPC A61B 5/02125; A61B 5/6813; A61B 5/7264; A61B 5/1116; A61B 5/1118; A61B 5/0022; A61B 2560/0238

(57) ABSTRACT

Provided is a method and apparatus to measure pulse wave information of a user. The method and apparatus obtain a basic blood pressure of the user estimated based on body information of a user. The method and apparatus determine a blood pressure calibration value corresponding to measured pulse wave information of the user using a pre-trained estimator. The method and apparatus calculate a final blood pressure of the user by applying the blood pressure calibration value to the basic blood pressure.

### CROSS-REFERENCE TO RELATED APPLICATION(S)

This application claims the benefit under 35 USC 119(a) of Korean Patent Application No. 10-2015-0181443, filed on Dec. 18, 2015, at the Korean Intellectual Property Office, the entire disclosure of which is incorporated herein by reference for all purposes.

### BACKGROUND

1. Field

The following description relates to a method and apparatus of calculating a blood pressure.

2. Description of Related Art

Recently, a method of using a pulse wave, for example, a pulse wave velocity (PWV) method and a pulse wave analysis (PWA) method, is used to measure a blood pressure in a user. The PWV method uses a time difference of pulse waves measured at two points after pressure sensors are disposed at different positions at artery blood vessels. The PWA method estimates a blood pressure based on a pulse wave.

Although the PWA and the PWV method are based on a high correlation in a blood pressure and elasticity of blood vessels, for these methods, calibration may be required to obtain a more accurate measurement of a blood pressure. Accurately measuring the blood pressure using an upper arm cuff sphygmomanometer and calibrating a blood pressure value of a cuffless sphygmomanometer based on the measured blood pressure may be used as a calibration method. However, such calibration method requires an additional cuff sphygmomanometer for accurate blood pressure calculation.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In accordance with an embodiment, there is provided a method of calculating a blood pressure, including: obtaining a basic blood pressure of the user estimated based on body information of a user; determining a blood pressure calibration value corresponding to measured pulse wave information of the user using a pre-trained estimator; and calculating a final blood pressure of the user by applying the blood pressure calibration value to the basic blood pressure.

The body information may include the pulse wave information.

The determining may include: extracting a feature from the pulse wave information; applying the feature to the estimator; and obtaining the blood pressure calibration value from the estimator.

The pulse wave information of the user may include at least one of a pulse wave according to a pulse wave analysis (PWA) and a time difference among pulse waves according to a pulse transition time (PTT).

The body information of the user may include at least one of a gender, an age, a height, a weight of the user, a body mass index (BMI) of the user, and posture information of the user.

The basic blood pressure may include at least one of an estimated value of a systolic blood pressure and an estimated value of a diastolic blood pressure.

The method may also include: providing a guide for the user to measure the pulse wave information at different positions; measuring the pulse wave information at the different positions; obtaining reference blood pressure calibration values corresponding to the different positions; and training the estimator based on the pulse wave information and the reference blood pressure calibration values.

The providing may include providing the guides showing the user how to assume postures.

The obtaining of the reference blood pressure calibration values may include calculating a hydrostatic pressure according to a height difference between a heart position of the user and a measurement position of the pulse wave information.

The training of the estimator may include determining a parameter of a regression equation, which estimates the blood pressure calibration value from the pulse wave information based on the pulse wave information measured at the different positions and the reference blood pressure calibration values.

The method may also include: estimating a posture of the user; measuring the pulse wave information corresponding to the posture; obtaining a reference blood pressure calibration value corresponding to the posture; and training the estimator based on the pulse wave information corresponding to the posture and the reference blood pressure calibration value.

The estimating of the posture of the user may include sensing a posture change during a daily activity of the user.

The obtaining may include calculating a hydrostatic pressure according to a height difference between a heart position of the user and a measurement position of the pulse wave information corresponding to the posture.

The training of the estimator may include determining a parameter of a regression equation, which estimates the blood pressure calibration value from the pulse wave information based on the reference blood pressure calibration value and the pulse wave information accumulated to correspond to postures.

The method may also include: transmitting at least one of the pulse wave information and the blood pressure calibration value to a server.

In accordance with an embodiment, there is provided a computer program embodied on a non-transitory computer readable medium, the computer program being configured to control a processor to perform the method described above.

In accordance with a further embodiment, there is provided an apparatus for calculating a blood pressure, including: a communication interface configured to receive body information of a user; and a processor configured to obtain a basic blood pressure of the user based on body information of the user, determine a blood pressure calibration value corresponding to measured pulse wave information of the user, and calculate a final blood pressure of the user based on the basic blood pressure and the blood pressure calibration value.

The processor may be configured to extract a feature from the pulse wave information, apply the feature to an estimator, and obtain the blood pressure calibration value from the estimator.

The apparatus may also include: a guide provider configured to provide a guide for the user such that the pulse wave information is measured at different positions, and wherein the processor is configured to measure the pulse wave information at the different positions, obtain reference blood pressure calibration values corresponding to the different positions, and train the estimator based on the pulse wave information and the reference blood pressure calibration values.

The processor may be configured to estimate a posture of the user using at least one of an acceleration sensor, an angular velocity sensor, and a gyro sensor, measure the pulse wave information corresponding to the posture, and train the estimator based on reference blood pressure calibration values obtained to correspond to the posture and the pulse wave information.

In accordance with an embodiment, there is provided a method, including: estimating, using an estimator, a basic blood pressure of a user including an absolute value of blood pressure based on body information of the user; and calibrating, using a processor, the basic blood pressure by applying a blood pressure calibration value to the basic blood pressure to obtain a final blood pressure, wherein the blood pressure value corresponds to pulse wave information of the user, and the pulse wave information may include a pulse wave and a time difference among pulse waves according to a pulse transition time.

The basic blood pressure may include at least one of an estimated value of a systolic blood pressure and an estimated value of a diastolic blood pressure.

The method may also include: measuring the pulse wave information at different positions of the user; obtaining reference blood pressure calibration values corresponding to each of the different positions; training the estimator based on the pulse wave information and the reference blood pressure calibration values; and outputting the blood pressure calibration value corresponding to the pulse wave information, wherein the reference blood pressure calibration values are blood pressure values calculated based on a hydrostatic pressure.

The method may also include: obtaining a change amount of the blood pressure based on the pulse wave information of the user based on a hydrostatic pressure.

Other features and aspects will be apparent from the following detailed description, the drawings, and the claims.

### BRIEF DESCRIPTION OF THE DRAWINGS

FIG. 1A is a block diagram illustrating an example of an apparatus to calculate a blood pressure, in accordance with an embodiment.

FIG. 1B illustrates an example of an operation of the apparatus to calculate the blood pressure, in accordance with an embodiment.

FIG. 2 is a flowchart illustrating an example of a method to calculate the blood pressure, in accordance with an embodiment.

FIG. 3 is a flowchart illustrating an example of a method to determine a blood pressure calibration value, in accordance with an embodiment.

FIG. 4 is a flowchart illustrating an example of a method to train an estimator, in accordance with an embodiment.

FIG. 5 is a flowchart illustrating another example of a method to train an estimator, in accordance with an embodiment.

FIG. 6 illustrates an example of a method to train an estimator based on a hydrostatic pressure, in accordance with an embodiment.

FIG. 7 illustrates an example of a method to calculate a blood pressure of a user based on a posture during a daily life activity of the user, in accordance with an embodiment.

FIG. 8 is a flowchart illustrating another example of a method to calculate a blood pressure, in accordance with an embodiment.

FIG. 9 is a block diagram illustrating another example of an apparatus to calculate the blood pressure, in accordance with an embodiment.

FIG. 10 is a block diagram illustrating still another example of an apparatus to calculate the blood pressure, in accordance with an embodiment.

Throughout the drawings and the detailed description, unless otherwise described or provided, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations described herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The following specific structural or functional descriptions are illustrative to merely describe the examples, and the scope of the examples is not limited to the descriptions provided in the present specification.

It will be understood that, although the terms first, second, etc. may be used herein to describe various elements, these elements should not be limited by these terms. These terms are only used to distinguish one element from another. For example, a first signal could be termed a second signal, and, similarly, a second signal could be termed a first signal without departing from the teachings of the disclosure.

It will be understood that when an element or layer is referred to as being “on”, “attached to”, or “connected to” another element or layer, it can be directly on or connected to the other element or layer or through intervening elements or layers may be present. In contrast, when an element is referred to as being “directly on”, “directly attached to”, or “directly connected to” another element or layer, there are no intervening elements or layers present. Other words used to describe the relationship between elements or layers should be interpreted in a like fashion (e.g., “between” versus “directly between,” “adjacent” versus “directly adjacent,” “on” versus “directly on”).

The terminology used herein is for the purpose of describing particular examples only and not to limit the examples. As used herein, the singular forms “a”, “an”, and “the” are intended to include the plural forms as well, unless the context clearly indicates otherwise. It will be further understood that the terms “include/comprise” and/or “have” when used in this specification, specify the presence of stated features, integers, steps, operations, elements, components, and/or combinations thereof, but do not preclude the presence or addition of one or more other features, numbers, steps, operations, elements, components, and/or groups thereof.

Unless otherwise defined, all terms including technical and scientific terms used herein have the same meaning as commonly understood by one of ordinary skill in the art to which examples belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

The following embodiments may be used for calculating a continuous blood pressure of a user during a daily life activity. Many of the embodiments described below may be implemented to be various forms, for example, a personal computer, a laptop computer, a tablet computer, a smartphone, a smart appliance, and a wearable device. Embodiments may be applied to obtain a basic blood pressure of a user, which is estimated based on body information of the user. A final blood pressure of the user is calculated by applying a pre-trained blood pressure calibration value, which corresponds to pulse wave information of the user, to the basic blood pressure through, for example, a smartphone, a mobile device, a smart home system, or a wearable device. Various embodiments may be also applied to, for example, a healthcare service for the user based on the calculated final blood pressure of the user. Hereinafter, reference will be made in detail to examples with reference to the accompanying drawings, wherein like reference numerals refer to like elements throughout. Embodiments may increase accuracy in calculating a blood pressure by blood pressure calibration based on a hydrostatic pressure, while continuously estimating a blood pressure from a biosignal including pulse wave information.

Hereinafter, a blood pressure is a pressure against a wall of a blood vessel while blood pumped from the heart is flowing through the blood vessel. A pulse wave is a wave formed when a pulse propagates through a peripheral arteriole. A pulse is a repetition of expansion and relaxation of an artery occurring due to a blood flow pushing blood along the artery at each heartbeat. Each time the heart contracts, blood is supplied to the entire body from the heart through an aorta, and a change in a pressure of a main artery occurs. Such a change in the pressure propagates to a peripheral arteriole of a hand and a foot, for instance, and is reflected as a waveform, such as, a pulse wave.

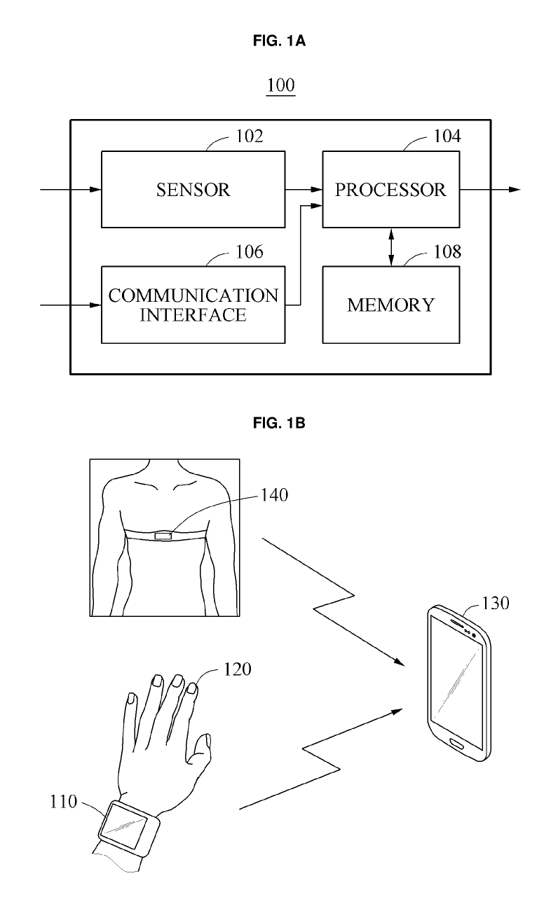

FIG. 1A is a block diagram illustrating an example of an apparatus to calculate a blood pressure, in accordance with an embodiment, and FIG. 1B illustrates an example of an operation of the apparatus to calculate a blood pressure, in accordance with an embodiment. FIG. 1A is a block diagram of the apparatus to calculate the blood pressure, hereinafter referred to as a calculation apparatus 100. FIG. 1B illustrates devices in which the calculation apparatus 100 is embedded.

Referring to FIG. 1A, the calculation apparatus 100 includes a sensor 102, a processor 104, a communication interface 106, and a memory 108. The sensor 102, the processor 104, the communication interface 106, and the memory 108 communicate with each other through a bus (not shown). The calculation apparatus 100 further includes an estimator (not shown) and a guide provider (not shown).

The sensor 102 measures or senses pulse wave information of a user. The sensor 102 includes a single sensor or a plurality of sensors. The sensor 102 includes a sensor to measure a photoplethysmogram (PPG), an electrocardiogram (ECG), a blood oxygen saturation (SpO2) level, and a ballistocardiogram (BCG), or a sensor to measure a change in a blood flow using an ultrasonic Doppler or a laser Doppler method. The sensor 102 includes an inertial sensor such as an acceleration sensor, a gyro sensor, a shock sensor, or a tilt sensor, or a global positioning system (GPS) sensor. However, embodiments are not limited thereto, and the sensor 102 may include various sensors in addition to or different from the sensors described in the foregoing paragraph.

The processor 104 obtains a basic blood pressure of the user estimated based on body information of the user. The basic blood pressure includes at least one of an estimated value of a systolic blood pressure and an estimated value of a diastolic blood pressure.

The processor 104 determines a blood pressure calibration value corresponding to the pulse wave information of the user using a pre-trained estimator (not shown). The processor 104 calculates a final blood pressure of the user based on the basic blood pressure and the blood pressure calibration value. The pulse wave information of the user includes, for example, at least one of a pulse wave, according to a pulse wave analysis (PWA), and a time difference among pulse waves, according to a pulse transition time (PTT).

The processor 104 extracts at least one feature from the pulse wave information and applies the at least one feature to the estimator. The processor 104 obtains the blood pressure calibration value from the estimator.

The communication interface 106 receives the body information of the user. The body information includes, for example, a gender, an age, a height, a weight of the user, a body mass index (BMI) of the user, posture information of the user, and the pulse wave information of the user. The BMI may be obtained by dividing a weight by a square of a height. In this example, a unit of the weight is a kilogram (kg) or a pound (Ib) and a unit of the square of the height is a square meter (m²) or a square foot (f²).

The guide provider (not shown) provides a guide for the user such that the pulse wave information is measured at a plurality of different positions.

The processor 104 measures the pulse wave information at the plurality of different positions and obtains reference blood pressure calibration values corresponding to each of the plurality of different positions. A reference blood pressure calibration value may be a blood pressure value calculated based on a hydrostatic pressure.

The processor 104 trains the estimator based on the reference blood pressure calibration values and the pulse wave information measured at the plurality of different positions.

The processor 104 estimates a posture of the user using at least one of, for example, an acceleration sensor, an angular velocity sensor, and a gyro sensor. The processor 104 measures the pulse wave information corresponding to the estimated posture and trains the estimator based on the pulse wave information corresponding to the posture and the reference blood pressure calibration values obtained to correspond to the estimated posture.

The memory 108 stores the final blood pressure calculated at the processor 104, the blood pressure calibration value, the basic blood pressure, and the body information including the pulse wave information of the user. The memory 108 may be a volatile memory or a non-volatile memory.

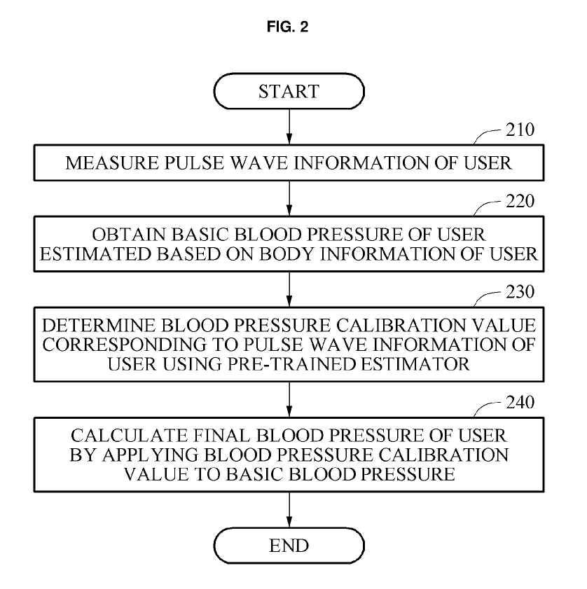

The processor 104 also performs at least one method described with reference to FIGS. 2 through 10. The processor 104 executes a program and controls the calculation apparatus 100. A program code executed by the processor 104 may be stored in the memory 108. The calculation apparatus 100 is connected to an external device, for example, a personal computer (PC), a mobile device, or a network through an input and output device (not shown) to perform a data exchange.

At least one of the methods described with reference to FIGS. 1 through 10 is implemented in an application executed in a processor of a tablet computer, a smartphone, or a wearable device, or implemented in a chip embedded in the smartphone or the wearable device.

FIG. 1B illustrates a wearable device 110 and a mobile device 130 in which the calculation apparatus 100 is to be embedded.

An operation of the calculation apparatus 100 embedded in the wearable device 110 will be explained. For example, the wearable device 110 is a wrist-worn device provided in a watch, a bracelet, and other similar wearable devices. Also, the wearable device 110 is provided as a necklace, bracelet, ring, or broach.

When a user 120 wearing the wearable device 110 on a wrist moves or sleeps, the calculation apparatus 100 estimates the basic blood pressure of the user 120 based on the pulse wave information measured from the wrist of the user 120 in addition to the body information of the user 120, and calculates the final blood pressure of the user 120 using the blood pressure calibration value corresponding to the pre-trained pulse wave information.

The wearable device 110 including the calculation apparatus 100 is interconnected with the mobile device 130 to share data with the mobile device 130. As an example, the pulse wave information measured from the user 120 or the blood pressure calibration value and the final blood pressure calculated by the calculation apparatus 100 are transferred to the mobile device 130.

In another example, the processor 104 of the calculation apparatus 100 is embedded in the mobile device 130, and the sensor 102 is embedded in the wearable device 110. The wearable device 110 is worn to be in contact with a body part, for example, a wrist of the user 120. The wearable device 110 measures a heart rate of the user 120 from the wrist as measured pulse wave information. The wearable device 110 amplifies and filters the measured pulse wave information. The wearable device 110 transmits the measured pulse wave information to the mobile device 130. The calculation apparatus 100 included in the mobile device 130 calculates the final blood pressure based on the measured pulse wave information received from the wearable device 110 and the blood pressure calibration value corresponding to the pulse wave information pre-stored in the mobile device 130.

The wearable device 110 is mutually connected with the mobile device 130 through a wireless link. The wearable device 110 and the mobile device 130 includes wireless Internet interfaces, such as a wireless local area network (WLAN) interface, Wi-Fi interface, a digital living network alliance (DLNA interface), a wireless broadband (WiBro) interface, a world interoperability for microwave access (WiMAX) interface, and a high-speed downlink packet access (HSDPA) interface, for example, and short-range communication interfaces, such as a Bluetooth interface, radio frequency identification (RFID) interface, infrared data association (IrDA) interface, a ultra wideband (UWB) interface, a ZigBee interface, and a near field communication (NFC) interface, for example.

The mobile device 130 is implemented as a tablet computer, a smart phone, or a personal digital assistant (PDA), for example. The mobile device 130 may be a type of network equipment, such as a server. The mobile device 130 may be a single server computer or a system similar thereto, or at least one server bank or server cloud distributed at different geographical locations.

The mobile device 130 receives various types of biosignals, as well as, the pulse wave information through the wearable device 110 or any other measuring device.

FIG. 2 is a flowchart illustrating an example of a method to calculate the blood pressure, in accordance with an embodiment. In operation 210, a calculation apparatus measures pulse wave information of a user. The pulse wave information includes, for example, a pulse wave according to a pulse wave analysis (PWA) and a time difference among pulse waves according to a pulse transition time (PTT).

In operation 220, the calculation apparatus obtains a basic blood pressure of the user, which is estimated based on body information of the user. The calculation apparatus estimates the basic blood pressure of the user based on the body information of the user. For example, in response to a photoplethysmogram (PPG) signal received from a sensor, the calculation apparatus analyzes a waveform of the PPG signal using the PWA, and estimates a blood pressure value, for example, a basic blood pressure, corresponding to the PPG signal using a preset blood pressure estimation model. Alternatively, the calculation apparatus receives the basic blood pressure of the user estimated based on the body information of the user by making a request to an outside source, external to the calculation apparatus. In this example, the basic blood pressure includes an estimated value of a systolic blood pressure and an estimated value of a diastolic blood pressure.

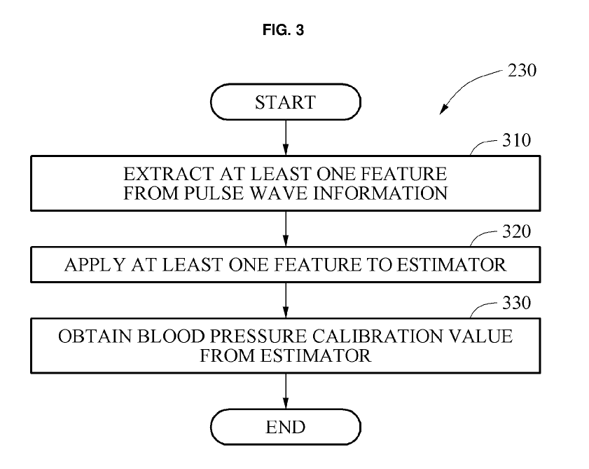

In operation 230, the calculation apparatus determines a blood pressure calibration value corresponding to the pulse wave information of the user using a pre-trained estimator. Descriptions related to a method in which a calculation apparatus determines a blood pressure calibration value will be provided with reference to FIG. 3.

In operation 240, the calculation apparatus calculates a final blood pressure of the user by applying the blood pressure calibration value to the basic blood pressure. The calculation apparatus calculates the final blood pressure by compensating, for example, calibrating the basic blood pressure of the user based on a blood pressure calibration value pre-trained to correspond to the pulse wave information of the user.

FIG. 3 is a flowchart illustrating an example of a method to determine a blood pressure calibration value, in accordance with an embodiment. In operation 310, a calculation apparatus extracts at least one feature from pulse wave information. In response to the pulse wave information of a pulse wave according to a pulse wave analysis (PWA), the calculation apparatus extracts a plurality of feature points as features based on the form of the pulse wave. In response to the pulse wave information being a time difference among pulse waves according to a pulse transition time (PTT), the calculation apparatus extracts a time difference, for example, 1 millisecond (ms) and 3 ms, as a feature.

In operation 320, the calculation apparatus applies the at least one feature extracted in operation 310 to an estimator. In operation 330, the estimator processes the at least one feature and outputs a blood pressure calibration value. In this example, the estimator is trained based on the pulse wave information and a reference blood pressure calibration value, and outputs the blood pressure calibration value corresponding to the pulse wave information. The reference blood pressure calibration value is determined based on a hydrostatic pressure. The hydrostatic pressure refers to a pressure in non-moving water or fluid, and the pressure at a single point of the fluid may have an identical value regardless of a direction of flow. A pressure P of the hydrostatic pressure may be obtained by a fluid density, p, times a gravitational acceleration, g, times a height (depth) h.

For example, in response to the pulse wave information being a form A of the pulse wave according to the PWA, the calculation apparatus applies the form A of the pulse wave to the estimator. In response, the estimator outputs an estimated value of a blood pressure that is pre-trained to correspond to the form A of the pulse wave. Alternatively, in response to the pulse wave information being a time difference 0.5 ms among pulse waves according to the PTT, the calculation apparatus applies the time difference 0.5 ms among the pulse waves to the estimator. In this example, the estimator outputs the estimated value of the blood pressure that is pre-trained to correspond to the time difference 0.5 ms among the pulse waves.

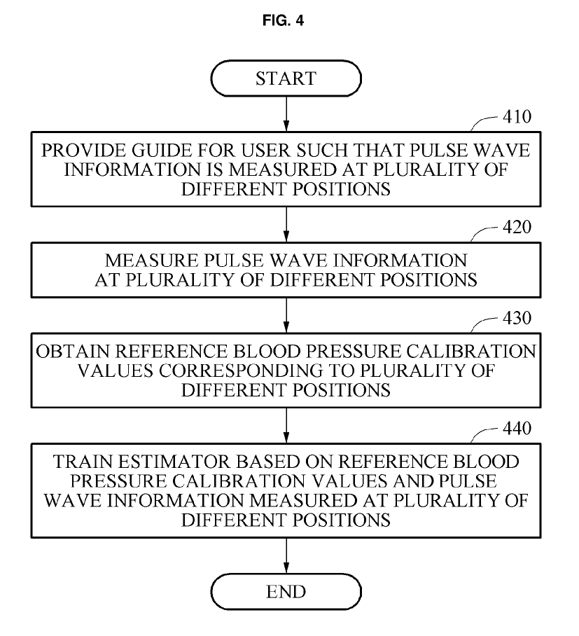

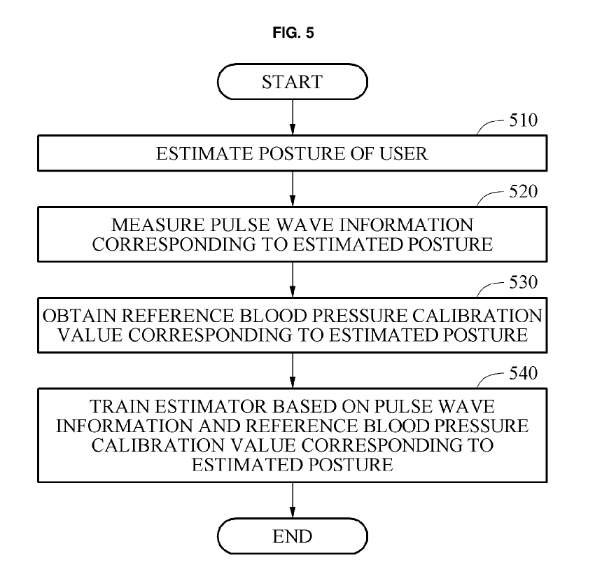

The calculation apparatus provides guides to show the user how to assume a plurality of different postures, trains the estimator by sensing a posture of the user based on the guide, and automatically trains the estimator by sensing a posture change during a daily life activity of the user. Hereinafter, an operation of training the estimator by sensing a posture of the user based on the guide will be referred to as “performing guided training”, and an operation that automatically trains the estimator by sensing a posture change during a daily life activity of the user will be referred to as “performing automatic training”. Descriptions related to a guided training method will be provided with reference to FIG. 4, and descriptions related to an automatic training method will be provided with reference to FIG. 5.

FIG. 4 is a flowchart illustrating an example of a method of training the estimator, in accordance with an embodiment. The example of FIG. 4 illustrates the method in which a calculation apparatus performs guided training on the estimator.

In operation 410, the calculation apparatus provides a guide for a user such that pulse wave information is measured at different positions. In one example, the calculation apparatus sequentially provides the guides to direct the user to assume different postures. The postures may be different postures that cause a difference between a heart position of the user and a measurement position of the pulse wave information. For example, when the position in which the pulse wave information is measured is at a wrist, the postures includes a posture of standing or sitting with an arm stretched upwards, a posture of standing at attention with hands lowered, a posture of standing or sitting with a hand held at a height of the heart, and a posture of lying down.

In operation 420, the calculation apparatus measures the pulse wave information at the different positions.

In operation 430, the calculation apparatus obtains reference blood pressure calibration values corresponding to the different positions. In this example, the calculation apparatus calculates a hydrostatic pressure according to a height difference between a heart position of the user and the measurement position of the pulse wave information. The calculation apparatus obtains the reference blood pressure calibration values corresponding to the different positions based on the hydrostatic pressure.

In operation 440, the calculation apparatus trains the estimator based on the reference blood pressure calibration values and the pulse wave information measured at the different positions. The calculation apparatus accumulates data based on blood pressure change values which are pre-calculated based on the hydrostatic pressure for each measurement positions of the pulse wave information, in addition to a pulse wave signal measured at an identical position. The calculation apparatus obtains the reference blood pressure calibration values by calculating a nerve network or a linear regression equation based on the data.

The calculation apparatus determines at least one parameter of a regression equation that estimates a blood pressure calibration value from the pulse wave information, based on the reference blood pressure calibration values and the pulse wave information measured at different positions.

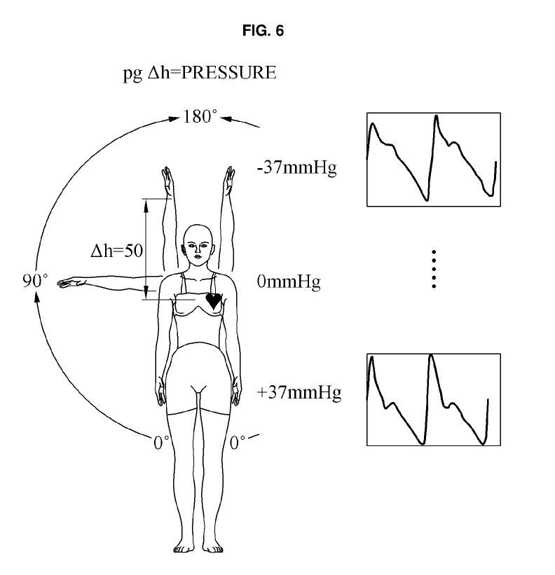

Descriptions related to a method in which a calculation apparatus trains an estimator, based on reference blood pressure calibration values that are obtained based on a hydrostatic pressure will be provided with reference to FIG. 6.

FIG. 5 is a flowchart illustrating another example of a method to train an estimator, in accordance with an embodiment. In operation 510, a calculation apparatus estimates a posture of a user. For example, the calculation apparatus estimates the posture of the user using an inertial sensor, such as temperature sensor and/or a global positioning system (GPS) sensor. For example, the calculation apparatus senses a posture change during a daily life activity of the user based on whether values of the acceleration sensor or the gyro sensor are fixed or drastically changed.

In operation 520, the calculation apparatus measures pulse wave information corresponding to the estimated posture.

In operation 530, the calculation apparatus obtains a reference blood pressure calibration value corresponding to the estimated posture. The calculation apparatus calculates a hydrostatic pressure using a height difference between a heart position of the user and a measurement position of the pulse wave information that corresponds to the estimated posture.

In operation 540, the calculation apparatus trains an estimator based on the pulse wave information that corresponds to the estimated posture measured in operation 520 and the reference blood pressure calibration value that corresponds to the estimated posture obtained in operation 530. The calculation apparatus determines at least one parameter of a regression equation that estimates a blood pressure calibration value from the pulse wave information. For instance, the calculation apparatus determines the at least one parameter of the regression equation based on the reference blood pressure calibration value and the pulse wave information accumulated to correspond to the postures.

FIG. 6 illustrates an example of a method of training the estimator based on a hydrostatic pressure, in accordance with an embodiment. A blood pressure changes over time and, thus, measuring a blood pressure only once, that is, at one time, may be insufficient to determine a state of a user. Thus, continuously measuring a blood pressure may be needed.

For example, where pulse wave information is measured on a wrist and a length of an arm of a user is 50 centimeters (cm), when the user raises a hand, a measurement position of the pulse wave information is higher than a height of a heart. Thus, a value of a blood pressure measured when the measurement position of the pulse wave information is higher than the height of the heart is decreased to be 37 millimeter of mercury (mmHg) by an error due to a height difference, for example, 50 cm, when compared to an actual value of a blood pressure. In response to the value of the blood pressure measured when the user assumes a posture of a hand raised being 90 mmHg, the calculation apparatus calculates a final blood pressure of the user based on calibration that adds a value of an error of 37 mmHg to the value of the blood pressure of 90 mmHg.

When the user assumes a posture of a hand lowered, the measurement position of the pulse wave information is lower than the height of the heart. Thus, a value of a blood pressure measured when the measurement position of the pulse wave information is lower than the height of the heart is increased to be +37 mmHg by an error due to the height difference when compared to the actual value of the blood pressure. In response to the value of the blood pressure measured when the user assumes a posture of the hand lowered being 132 mmHg, the calculation apparatus calculates the final blood pressure of the user based on the calibration that subtracts the value of the error of 37 mmHg from the value of the blood pressure of 132 mmHg.

In an example, when compensation is performed on a basic blood pressure of the user based on the pulse wave information, for example, when a form change in a waveform or a value change of 1 millisecond (msec) occurs in the pulse wave information, the calculation apparatus may be unable to have information about a scale factor. In this example, the scale factor indicates whether such value change is to be converted into a blood pressure of 10 mmHg or a blood pressure of 20 mmHg.

The calculation apparatus calculates a nerve network or a regression equation, for example, a hydrostatic pressure Y is equal to a change amount of pulse wave information (aX+b), which estimates a blood pressure calibration value from the pulse wave information, based on the pulse wave information measured at a plurality of positions, and reference blood pressure calibration values, which are pre-calculated based on the hydrostatic pressure for each of measurement positions of the pulse wave information. The calculation apparatus determines at least one of parameters, a and b, from the calculated regression equation and uses the parameter, a, which corresponds to a gradient of the regression equation, as the scale factor of the pulse wave information. The calculation apparatus obtains a change amount of the blood pressure based on the pulse wave information of the user based on the hydrostatic pressure.

In an example, a pulse wave during a change of the hydrostatic pressure occurring due to the height difference between the heart position and the measurement position of the pulse wave are measured and the final blood pressure of the user is accurately calculated by pre-training an estimator to have a blood pressure calibration value corresponding to the measured form of the pulse wave.

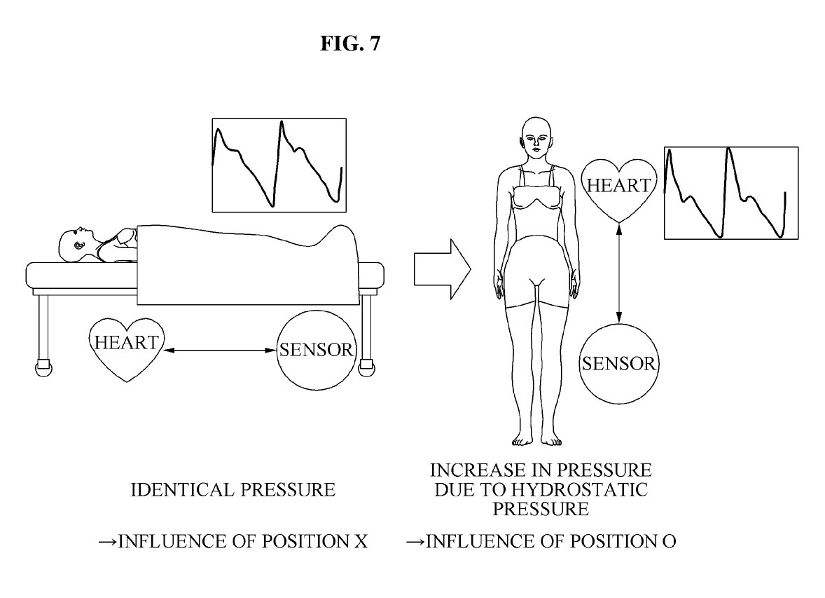

FIG. 7 illustrates an example of a method of calculating a blood pressure of a user based on a posture during a daily life activity of the user, in accordance with an embodiment. Referring to FIG. 7, a calculation apparatus verifies that a user is sleeping or doing an activity using, for example, various inertia sensors and a global positioning system (GPS) sensor. In such an example, the calculation apparatus verifies whether a basic blood pressure of the user is influenced by a position due to a height difference or a distance difference between a heart position of the user and a measurement position of pulse wave information. Thus, when the height difference between the heart position of the user and the measurement position of the pulse wave information is not present, the calculation apparatus outputs the basic blood pressure as a final blood pressure based on a determination that an identical pressure is at the heart position and the measurement position of the pulse wave information.

In response to an occurrence of a height difference between the heart position of the user and the measurement position of the pulse wave information, the calculation apparatus determines that the basic blood pressure is influenced by the position because an increase or decrease in the pressure occurs due to a hydrostatic pressure. The calculation apparatus calculates the final blood pressure by calibrating the basic blood pressure by a blood pressure calibration value pre-trained to correspond to the pulse wave information.

The calculation apparatus continuously transmits a blood pressure to a server by naturally and continuously measuring the blood pressure during a daily life activity

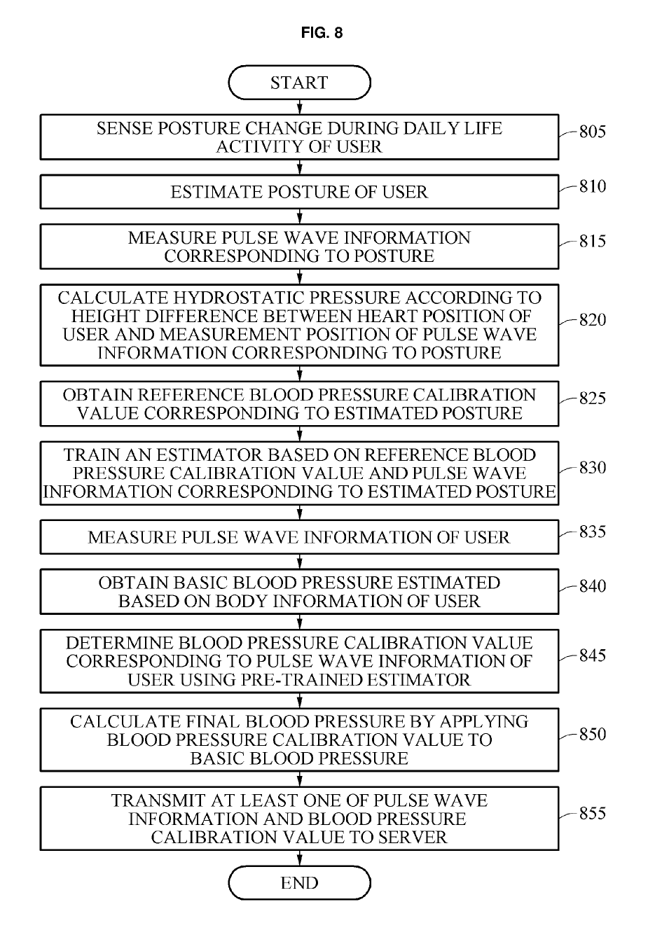

FIG. 8 is a flowchart illustrating another example of a method of calculating a blood pressure, in accordance with an embodiment. In operation 805, a calculation apparatus senses at least one posture change during a daily life activity of a user.

In operation 810, the calculation apparatus estimates a current posture of the user. In accordance with an alternative embodiment, although the calculation apparatus is estimating the current posture of the user, the calculation apparatus is also configured to estimate past postures of the user and continuously calculate the blood pressure activity during a preset period of time.

In operation 815, the calculation apparatus measures pulse wave information corresponding to the current posture estimated in operation 810.

In operation 820, the calculation apparatus calculates a hydrostatic pressure according to a height difference between a heart position of the user and a measurement position of the pulse wave information corresponding to the current posture.

In operation 825, the calculation apparatus obtains a reference blood pressure calibration value corresponding to the estimated posture.

In operation 830, the calculation apparatus trains an estimator based on the reference blood pressure calibration value and the pulse wave information corresponding to the estimated posture.

In operation 835, the calculation apparatus measures the pulse wave information of the user.

In operation 840, the calculation apparatus obtains a basic blood pressure estimated based on body information of the user.

In operation 845, the calculation apparatus determines a blood pressure calibration value corresponding to the pulse wave information of the user using a pre-trained estimator.

In operation 850, the calculation apparatus calculates a final blood pressure by applying the blood pressure calibration value to the basic blood pressure.

In operation 855, the calculation apparatus transmits at least one of the pulse wave information and the blood pressure calibration value to a server.

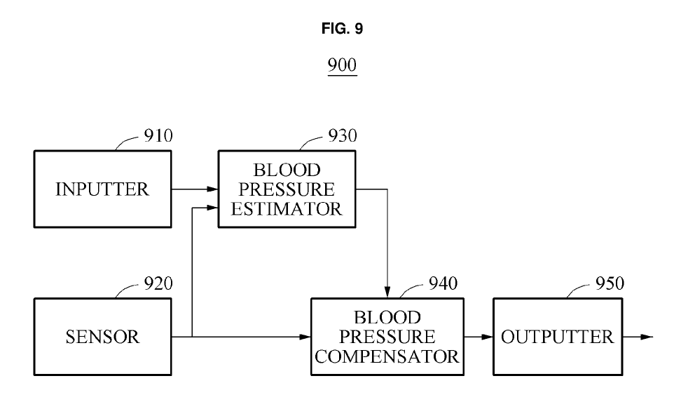

FIG. 9 is a block diagram illustrating another example of an apparatus to calculate a blood pressure, in accordance with an embodiment. A calculation apparatus 900 includes an inputter 910, a sensor 920, a blood pressure estimator 930, a blood pressure compensator 940, and an outputter 950.

The inputter 910 receives body information of a user.

The sensor 920 continuously measures pulse wave information of the user.

The blood pressure estimator 930 estimates a basic blood pressure of the user based on, for example, the body information and the pulse wave information of the user measured by the sensor 920. In an example, the blood pressure estimator 930 estimates the basic blood pressure of the user using statistical variables or a preset blood pressure estimation model.

The blood pressure compensator 940 performs compensation by applying the continuously measured pulse wave information to the basic blood pressure of the user estimated by the blood pressure estimator 930.

The outputter 950 outputs, as the final blood pressure, a blood pressure onto which the compensation is performed.

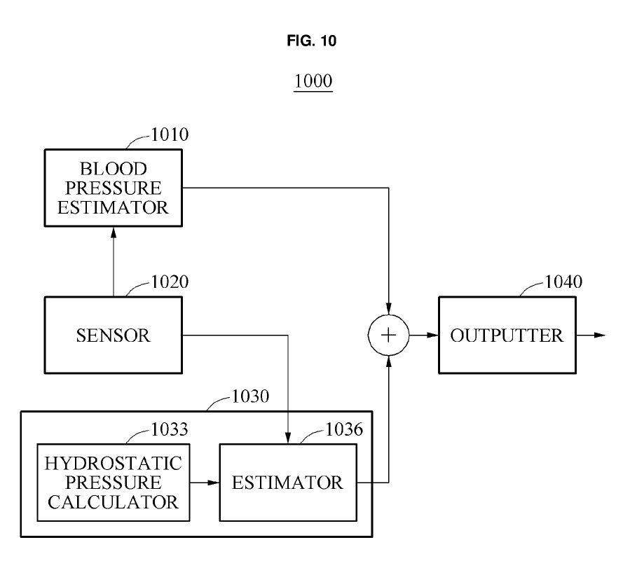

FIG. 10 is a block diagram illustrating still another example of an apparatus to calculate a blood pressure, in accordance with an embodiment. Referring to FIG. 10, a calculation apparatus 1000 includes a blood pressure estimator 1010, a sensor 1020, a blood pressure compensator 1030, and an outputter 1040. The blood pressure compensator 1030 includes a hydrostatic pressure calculator 1033 and an estimator 1036.

The blood pressure estimator 1010 estimates a basic blood pressure of a user based on body information of the user and pulse wave information of the user measured by the sensor 1020.

The sensor 1020 measures the pulse wave information of the user.

The hydrostatic pressure calculator 1033 calculates a hydrostatic pressure according to a height difference between a heart position of the user and a measurement position of the pulse wave information.

The estimator 1036 estimates a blood pressure calibration value corresponding to the pulse wave information. In accordance with an embodiment, the estimator 1036 is pre-trained by a regression equation that estimates the blood pressure calibration value corresponding to the pulse wave information. The regression equation is based on reference blood pressure calibration values and a pulse wave when the hydrostatic pressure occurring, due to a height difference between the heart position and the measurement position, changes.

The blood pressure compensator 1030 performs compensation on the basic blood pressure that the blood pressure estimator 1010 estimated using the blood pressure calibration value that the estimator 1036 estimates.

The outputter 1050 outputs a final blood pressure onto which the compensation is performed in the blood pressure compensator 1030.

The calculation apparatus 1000 estimates a blood pressure value based on the hydrostatic pressure, without having a need to use an additional cuff sphygmomanometer. The calculation apparatus 1000 collects the measured hydrostatic pressure and the pulse wave information, for example, a pulse wave, corresponding to a position at which the hydrostatic pressure is calculated.

In accordance with an embodiment, the calculation apparatus 1000 accurately calculates a change in an amount of a blood pressure according to each pulse wave feature of the user by the estimator 1036, using a process or a method that provides the blood pressure calibration value corresponding to the pulse wave information through training.

In FIGS. 1A, 9, and 10, although certain structural elements are illustrated and described, a person of ordinary skill in the relevant art will appreciate that some of the elements may be external to the calculation apparatus, without departing from the core of the embodiments. For instance, the sensor illustrated and described may be external to the calculation apparatus.

The apparatuses, interfaces, inputters, outputters, calculators, estimators, compensators, sensors, devices, and other components illustrated in FIGS. 1A, 9, and 10 that perform the operations described herein with respect to FIGS. 2-5 and 8 are implemented by hardware components. Examples of hardware components include processors, controllers, sensors, generators, drivers, and any other electronic components known to one of ordinary skill in the art. In one example, the hardware components are implemented by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of calculating a blood pressure, comprising:
obtaining a basic blood pressure of the user estimated based on body information of a user;
determining a blood pressure calibration value corresponding to measured pulse wave information of the user using a pre-trained estimator; and
calculating a final blood pressure of the user by applying the blood pressure calibration value to the basic blood pressure.

2. The method of claim 1, wherein the body information comprises the pulse wave information.

3. The method of claim 1, wherein the determining comprises:
extracting a feature from the pulse wave information;
applying the feature to the estimator; and
obtaining the blood pressure calibration value from the estimator.

4. The method of claim 1, wherein the pulse wave information of the user comprises at least one of a pulse wave according to a pulse wave analysis (PWA) and a time difference among pulse waves according to a pulse transition time (PTT).

5. The method of claim 1, wherein the body information of the user comprises at least one of a gender, an age, a height, a weight of the user, a body mass index (BMI) of the user, and posture information of the user.

6. The method of claim 1, wherein the basic blood pressure comprises at least one of an estimated value of a systolic blood pressure and an estimated value of a diastolic blood pressure.

7. The method of claim 1, further comprising:
providing a guide for the user to measure the pulse wave information at different positions;
measuring the pulse wave information at the different positions;
obtaining reference blood pressure calibration values corresponding to the different positions; and
training the estimator based on the pulse wave information and the reference blood pressure calibration values.

8. The method of claim 7, wherein the providing comprises providing the guides showing the user how to assume postures.

9. The method of claim 7, wherein the obtaining of the reference blood pressure calibration values comprises calculating a hydrostatic pressure according to a height difference between a heart position of the user and a measurement position of the pulse wave information.

10. The method of claim 7, wherein the training of the estimator comprises determining a parameter of a regression equation, which estimates the blood pressure calibration value from the pulse wave information based on the pulse wave information measured at the different positions and the reference blood pressure calibration values.

11. The method of claim 1, further comprising:
estimating a posture of the user;
measuring the pulse wave information corresponding to the posture;
obtaining a reference blood pressure calibration value corresponding to the posture; and
training the estimator based on the pulse wave information corresponding to the posture and the reference blood pressure calibration value.

12. The method of claim 11, wherein the estimating of the posture of the user comprises sensing a posture change during a daily activity of the user.

13. The method of claim 12, wherein the obtaining comprises calculating a hydrostatic pressure according to a height difference between a heart position of the user and a measurement position of the pulse wave information corresponding to the posture.

14. The method of claim 12, wherein the training of the estimator comprises determining a parameter of a regression equation, which estimates the blood pressure calibration value from the pulse wave information based on the reference blood pressure calibration value and the pulse wave information accumulated to correspond to postures.

15. The method of claim 1, further comprising:
transmitting at least one of the pulse wave information and the blood pressure calibration value to a server.

16. A computer program embodied on a non-transitory computer readable medium, the computer program being configured to control a processor to perform the method of claim 1.

17. An apparatus for calculating a blood pressure, comprising:
a communication interface configured to receive body information of a user; and
a processor configured to obtain a basic blood pressure of the user based on body information of the user, determine a blood pressure calibration value corresponding to measured pulse wave information of the user, and calculate a final blood pressure of the user based on the basic blood pressure and the blood pressure calibration value.

18. The apparatus of claim 17, wherein the processor is configured to extract a feature from the pulse wave information, apply the feature to an estimator, and obtain the blood pressure calibration value from the estimator.

19. The apparatus of claim 17, further comprising:
a guide provider configured to provide a guide for the user such that the pulse wave information is measured at different positions, and
wherein the processor is configured to measure the pulse wave information at the different positions, obtain reference blood pressure calibration values corresponding to the different positions, and train the estimator based on the pulse wave information and the reference blood pressure calibration values.

20. The apparatus of claim 17, wherein the processor is configured to estimate a posture of the user using at least one of an acceleration sensor, an angular velocity sensor, and a gyro sensor, measure the pulse wave information corresponding to the posture, and train the estimator based on reference blood pressure calibration values obtained to correspond to the posture and the pulse wave information.

21. A method, comprising:
estimating, using an estimator, a basic blood pressure of a user comprising an absolute value of blood pressure based on body information of the user; and
calibrating, using a processor, the basic blood pressure by applying a blood pressure calibration value to the basic blood pressure to obtain a final blood pressure, wherein the blood pressure value corresponds to pulse wave information of the user, and the pulse wave information comprises a pulse wave and a time difference among pulse waves according to a pulse transition time.

22. The method of claim 21, wherein the basic blood pressure comprises at least one of an estimated value of a systolic blood pressure and an estimated value of a diastolic blood pressure.

23. The method of claim 21, further comprising:
measuring the pulse wave information at different positions of the user;
obtaining reference blood pressure calibration values corresponding to each of the different positions;
training the estimator based on the pulse wave information and the reference blood pressure calibration values; and
outputting the blood pressure calibration value corresponding to the pulse wave information, wherein the reference blood pressure calibration values are blood pressure values calculated based on a hydrostatic pressure.

24. The method of claim 21, further comprising:
obtaining a change amount of the blood pressure based on the pulse wave information of the user based on a hydrostatic pressure.
